(19) United States
(12) Patent Application Publication
(10) Pub. No.: US 2026/0248744 A1
(43) Pub. Date: Aug. 27, 2026

(54) USE OF NON-TOXIC POLYAMINE ANALOGUES AND/OR INHIBITORS OF POLYAMINE BIOSYNTHESIS TO RE-BALANCE NATURAL POLYAMINE LEVELS IN SNYDER-ROBINSON SYNDROME AND RELATED DISORDERS

(71) Applicant: The Johns Hopkins University, Baltimore, MD (US)

(72) Inventors: Robert A. CASERO, Baltimore, MD (US); Tracy Murray STEWART, Baltimore, MD (US)

(21) Appl. No.: 18/872,474

(22) PCT Filed: Jun. 19, 2023
(86) PCT No.: PCT/US2023/025659

§ 371 Date: Dec. 6, 2024

Related U.S. Application Data
(60) Provisional application No. 63/353,711, filed on Jun. 20, 2022.

(51) Int. Cl. A61K 31/132; A61K 31/198; A61K 31/7076; A61K 45/06; A61P 3/00

(52) U.S. Cl. CPC A61K 31/132; A61K 31/198; A61K 31/7076; A61K 45/06; A61P 3/00

(57) ABSTRACT

Methods for treating Snyder-Robinson Syndrome or related disorders by administering a polyamine analogue in combination with an inhibitor of polyamine biosynthesis are disclosed.

### FEDERALLY SPONSORED RESEARCH OR DEVELOPMENT

This invention was made with government support under grants CA235863 and CA204345 awarded by the National Institutes of Health. The government has certain rights in the invention.

### BACKGROUND

Snyder-Robinson Syndrome (SRS) is an X-linked intellectual disability disorder caused by a loss-of function mutation in the spermine synthase (SMS) gene. SRS occurs primarily in males. Common symptoms include intellectual disability, developmental delay, including delays and/or deficits in speech, mobility, and cognition, osteoporosis, hypotonia (progressive), scoliosis/kyphosis, and seizures. Less common symptoms include speech and gait abnormalities, facial asymmetry, cleft palate, prominent lower lip, renal abnormalities, pulmonary/respiratory infections, and immunological abnormalities. Typical treatment includes anti-seizure medications, calcium supplements, and speech and physical therapy. The biochemical phenotype of SRS-affected cells and tissues include a decrease in spermine synthase (SMS) activity; an increase in spermidine (SPD) levels; a decrease in spermine (SPM) levels; and an increase in the SPD/SPM ratio.

### SUMMARY

The presently disclosed subject matter provides methods for treating a disease, condition, or disorder associated with a decrease in biosynthesis of spermine and/or an elevated spermidine/spermine level, the method comprising administering to a subject in need of treatment thereof a therapeutically effective amount of a polyamine analogue or an inhibitor of polyamine biosynthesis, such as a an ornithine decarboxylase (ODC) inhibitor or a spermidine synthase (SRM) inhibitor, or a combination thereof, to treat disease, condition, or disorder.

In some aspects, the disease, condition, or disorder associated with a decrease in biosynthesis of spermine and/or an elevated spermidine/spermine level comprises Snyder-Robinson Syndrome or related disorders.

In certain aspects, the polyamine analogue comprises a spermine mimetic. In particular aspects, the spermine mimetic is selected from (R,R)-1,12-dimethylspermine, (S,S)-1,12-dimethylspermine, (R,S)-1,12-dimethylspermine, 3,10-dimethylspermine and 2,11-dimethylspermine. In particular aspects, the spermine mimetic comprises 1,12-dimethylspermine. In more particular aspects, the 1,12-dimethylspermine comprises (R,R)-1,12-dimethylspermine.

In certain aspects, the inhibitor of polyamine biosynthesis is an ornithine decarboxylase (ODC) inhibitor selected from difluoromethylornithine (DFMO, Eflornithine), alpha-methylornithine, alpha(fluoromethyl)dehydroornithine, alpha-(fluoromethyl)dehydroputrescine, N-ω-chloroacetyl-L-ornithine, and alpha-allenyl putrescine.

In certain aspects, the inhibitor of polyamine biosynthesis is a spermidine synthase (SRM) inhibitor selected from decarboxylated S-adenosylhomocysteine (dcSAH), bis-cyclohexylammonium sulfate (BCHS), Juglorin, adenosyl spermidine, S-adenosyl-1,8-diamino-3-thio-octane. (AdoDATO), trans-4-methylcyclohexylamine (4MCHA), cyclohexylamine (CHA), N-(3-Aminopropyl)cyclohexylamine, dicyclohexylamine (DCHA) sulfate, methylglyoxal bis-(cyclopentylamidinohydrazone) (MGBP), 2-mercaptoethylamine, 2-mercaptopropylamine, N-chlorosulfonyldicyclohexylamine, 5′-((3-aminopropyl)ammo)-5′-deoxyadenosine, 1-aminooxy-3-aminopropane, 5′-(isobutylthio)adenosine, and 5′-(methylthio)adenosine.

In certain aspects, administration of the polyamine analogue in combination with an inhibitor of polyamine biosynthesis restores a balance of polyamine levels in the subject relative to a balance of polyamine levels in a subject not afflicted with disease, condition, or disorder associated with a decrease in biosynthesis of spermine and/or an elevated spermidine/spermine level, such as Snyder-Robinson Syndrome or related disorders.

In certain aspects, administration of the polyamine analogue in combination with an inhibitor of polyamine biosynthesis results in one or more of an increase in spermine synthase (SMS) activity, a decrease in intracellular spermidine (SPD), an increase in intracellular spermine (SPM), and a decrease in SPD/SPM ratio.

In other aspects, the presently disclosed subject matter provides a composition comprising a polyamine analogue and an inhibitor of polyamine biosynthesis and a pharmaceutically acceptable excipient.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 is a schematic representation of the effects of spermine synthase (SMS) dysfunction on polyamine metabolism (adapted from Murray-Stewart et al., 2018);

FIG. 2 demonstrates that long-term in vivo exposure to low-dose (R,R)-1,12-Me₂Spm is nontoxic and improves the SPD/SPM ratio in WT male C57Bl/6J mice;

FIG. 3 is a schematic representation of the hypothesis that inhibiting polyamine biosynthesis will enhance uptake of (R,R)-1,12-Me₂Spm and/or reduce the dose necessary for spermidine reduction;

FIG. 4A, FIG. 4B, and FIG. 4C demonstrate that DFMO and Me₂SPM cooperatively reduce intracellular spermidine, improve SPD/SPM ratio, and support proliferation in cells from SRS patients. (FIG. 4A) DFMO increases uptake of Me₂Spm. (FIG. 4B) DFMO cooperates with Me₂Spm in improving SPD/SPM ratio, indicated by numbers above columns. (FIG. 4C) Me₂Spm rescues DFMO-mediated growth inhibition. In FIG. 4A and FIG. 4B, SRS patient-derived lymphoblastoid cells were pretreated with DFMO followed by 96-h cotreatment with DFMO and Me₂Spm. SRS fibroblast lines in FIG. 4C were cotreated with DFMO and Me₂Spm for 96 h;

FIG. 5A, FIG. 5B, FIG. 5C, FIG. 5D, and FIG. 5E show potential therapeutic targets and molecules in the response of SRS cells to DFMO. DFMO enhances conversion of SPD to SPM (FIG. 5A), correcting the SPD/SPM ratio (FIG. 5B) in SRS patient-derived cell lines. DFMO induces SAMDC activity (FIG. 5C), and cotreatment with a SAMDC inhibitor (CGP48664) prevents the conversion to SPM (FIG. 5D), suggesting increased availability of the aminopropyl donor dcSAM to the hypomorphic SMS enzyme as a mechanism of action. SRS cells with a complete loss-of-function mutation in SMS do not convert SPD to SPM (FIG. 5E), indicating stimulation of hypomorphic SMS enzyme activity in the response to DFMO; and

FIG. 6 illustrates SRM as a second therapeutic target in the polyamine biosynthetic pathway. AdoDATO inhibits spermidine synthase (SRM), thereby preventing SPD biosynthesis and preserving dcSAM pool for conversion of SPD to SPM by hypomorphic SMS. Treatment of SRS cells with AdoDATO improves the SPD/SPM ratio while maintaining levels of putrescine, which DFMO depletes.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides methods for treating a disease, condition, or disorder associated with a decrease in biosynthesis of spermine and/or an elevated spermidine/spermine level, the method comprising administering to a subject in need of treatment thereof a therapeutically effective amount of a polyamine analogue or an inhibitor of polyamine biosynthesis, such as a an ornithine decarboxylase (ODC) inhibitor or a spermidine synthase (SRM) inhibitor, or a combination thereof, to treat disease, condition, or disorder.

In certain embodiments, the disease, condition, or disorder associated with a decrease in biosynthesis of spermine and/or an elevated spermidine/spermine level comprises Snyder-Robinson Syndrome or related disorders.

The natural polyamines include putrescine (1,4-diaminobutane), spermidine (1,8-diamino-4-azaoctane) and spermine (1,12-diamino-4,9-diazadodecane):

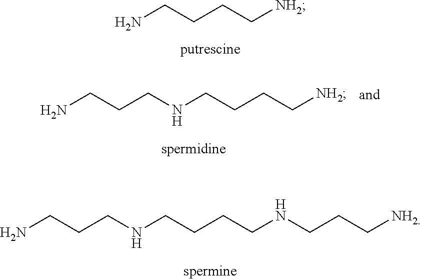 [SMILES: NCCCCN ; NCCCCNCCCN ; NCCCNCCCCNCCCN]

Representative polyamine analogues are disclosed in Casero and Woster, 2009, which is incorporated herein by reference in its entirety. Additional polyamine analogues are disclosed in U.S. Pat. No. 7,208,528 for Polyamine analogues as therapeutic and diagnostic agents, to Vermeulin et al., issued Apr. 24, 2007, which is incorporated herein by reference in its entirety.

In certain embodiments, the polyamine analogue comprises a spermine mimetic. Representative spermine mimetics include, but are not limited to, (R,R)-1,12-dimethylspermine, (S,S)-1,12-dimethylspermine, (R,S)-1,12-dimethylspermine, 3,10-dimethylspermine and 2,11-dimethylspermine. See RU2558953C2 for (R,R)- and (S,S)-diastereomers of 2,11-dimethylspermine and 3,10-dimethylspermine, to Khomutov et al., published Aug. 10, 2015, which is incorporated herein by reference in its entirety. In particular embodiments, the spermine mimetic comprises 1,12-dimethylspermine. In more particular embodiments, the 1,12-dimethylspermine comprises (R,R)-1,12-dimethylspermine.

In certain embodiments, the inhibitor of polyamine biosynthesis comprises an ornithine decarboxylase (ODC) inhibitor selected from difluoromethylornithine (DFMO), alpha-methylornithine, alpha(fluoromethyl)dehydroornithine, alpha-(fluoromethyl)dehydroputrescine, N-ω-chloroacetyl-L-ornithine, and alpha-allenyl putrescine. See, for example, Bey et al., 1983; Medina-Enríquez et al., 2015; and Danzin and Casara, 1984.

In certain embodiments, the inhibitor of polyamine biosynthesis is a spermidine synthase (SRM) inhibitor selected from decarboxylated S-adenosylhomocysteine (dcSAH), see Sečkutė et al., 2022, bis-cyclohexylammonium sulfate (BCHS), Juglorin, adenosyl spermidine, S-adenosyl-1,8-diamino-3-thio-octane. (AdoDATO), trans-4-methylcyclohexylamine (4MCHA), cyclohexylamine (CHA), N-(3-Aminopropyl)cyclohexylamine, dicyclohexylamine (DCHA) sulfate, methylglyoxal bis-(cyclopentylamidinohydrazone) (MGBP), 2-mercaptoethylamine, 2-mercaptopropylamine, N-chlorosulfonyldicyclohexylamine, 5′-((3-aminopropyl)ammo)-5′-deoxyadenosine, 1-aminooxy-3-aminopropane, 5′-(isobutylthio)adenosine, and 5′-(methylthio)adenosine.

In certain embodiments, administration of the polyamine analogue in combination with an inhibitor of polyamine biosynthesis restores a balance of polyamine levels in the subject relative to a balance of polyamine levels in a subject not afflicted with the disease, condition, or disorder associated with a decrease in biosynthesis of spermine and/or an elevated spermidine/spermine level, such as Snyder-Robinson Syndrome or related disorders.

In certain embodiments, administration of the polyamine analogue in combination with an inhibitor of polyamine biosynthesis results in one or more of an increase in spermine synthase (SMS) activity, a decrease in intracellular spermidine (SPD), an increase in intracellular spermine (SPM), and a decrease in SPD/SPM ratio.

In other embodiments, the presently disclosed subject matter provides a composition comprising a polyamine analogue and an inhibitor of polyamine biosynthesis and a pharmaceutically acceptable excipient.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the drug target, and the like.

The term “combination” is used in its broadest sense and means that a subject is administered at least two agents, more particularly a polyamine analogue in combination with an inhibitor of polyamine biosynthesis. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the polyamine analogue in combination with an inhibitor of polyamine biosynthesis can be further administered with adjuvants that enhance stability of the agents, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of polyamine analogue in combination with an inhibitor of polyamine biosynthesis can be varied so long as the beneficial effects of the combination of these agents are achieved. Accordingly, the phrase “in combination with” refers to the administration of a polyamine analogue described herein and an inhibitor of polyamine biosynthesis either simultaneously, sequentially, or a combination thereof. Therefore, a subject administered a combination of a polyamine analogue and an inhibitor of polyamine biosynthesis can receive a polyamine analogue and an inhibitor of polyamine biosynthesis or at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the polyamine analogue and an inhibitor of polyamine biosynthesis are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + B / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Use of Non-Toxic Polyamine Analogues and/or Inhibitors of Polyamine Biosynthesis to Re-Balance Natural Polyamine Levels in Snyder-Robinson Syndrome and Related Disorders

### 1.1 Overview

The presently disclosed subject matter provides a new drug strategy to treat individuals affected by the genetic disease known as Snyder-Robinson Syndrome, which results from mutations in or loss of the spermine synthase gene and results in a severe imbalance in intracellular concentrations of the natural polyamines.

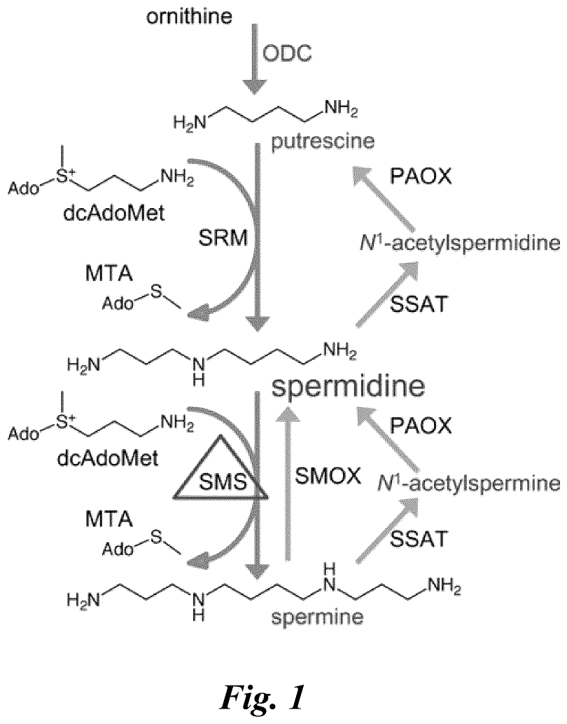

The effects of spermine synthase (SMS) dysfunction on polyamine metabolism are shown in FIG. 1 (adapted from Murray-Stewart et al., 2018). As provided in FIG. 1, the diamine PUT is derived from ornithine via ODC, the first rate-limiting step in polyamine biosynthesis. Sequential aminopropyl group additions by spermidine synthase (SRM) and SMS, using decarboxylated S-adenosylmethionine (dcAdoMet) as a donor, create SPD and SPM, respectively. Catabolismof SPM and SPD occurs via acetylation by SSAT followed by oxidation by PAOX. SPM can also be directly oxidized to SPD by SMOX. In SRS, SMS function is decreased or absent, spermidine (SPD) accumulates, and spermine (SPM) levels are severely reduced, resulting in an increase in the SPD/SPM ratio.

(R,R)-1,12-dimethylspermine has been shown previously to be a metabolically stable spermine mimetic (Murray Stewart et al., 2020):

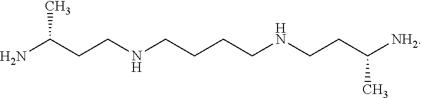 [SMILES: C[C@@H](N)CCNCCCCNCC[C@@H](C)N]

1,12-dimethylspermine also can exist as the (S,S) diastereomer:

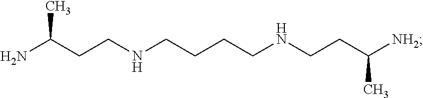 [SMILES: C[C@H](N)CCNCCCCNCC[C@H](C)N]

see RU2558953C2 for (R,R)- and (S,S)-diastereomers of 2,11-dimethylspermine and 3,10-dimethylspermine, to Khomutov et al., published Aug. 10, 2015, which is incorporated herein by reference in its entirety.

### 1.2 Results and Discussion

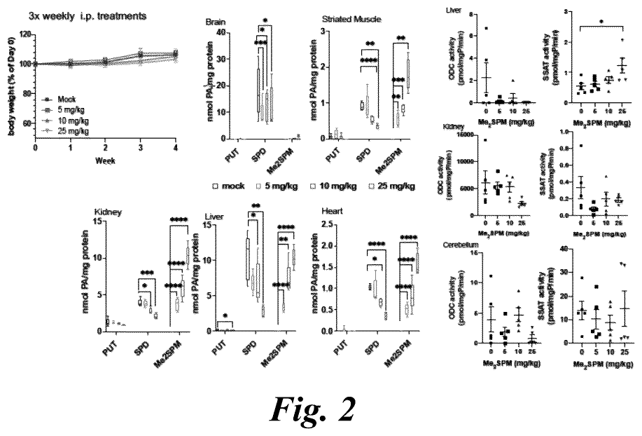

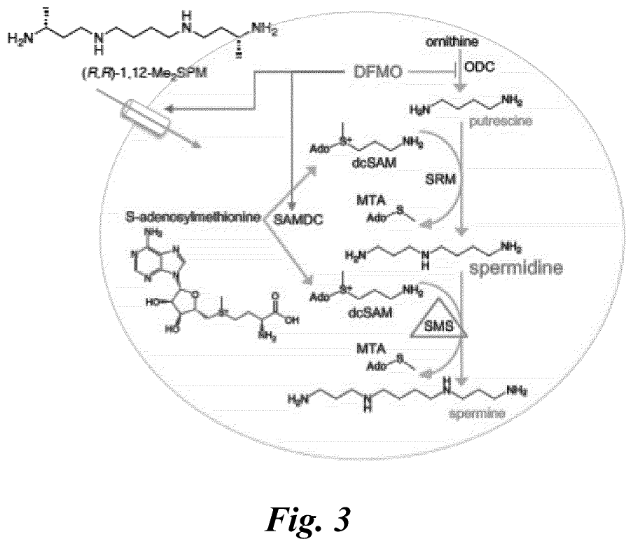

Long-term in vivo exposure to low-dose (R,R)-1,12-dimethylspermine is nontoxic and improves the SPD/SPM ratio in WT male C57Bl/6J mice (see FIG. 2). Without wishing to be bound to any one particular theory, it is thought that inhibiting polyamine biosynthesis will enhance uptake of 1,12-Me₂Spm and/or reduce the dose necessary for spermidine reduction.

Difluoromethylornithine (DFMO), also referred to as “eflornithine,” is an irreversible inhibitor of ornithine decarboxylase (ODC) with consequential upregulation of polyamine uptake and S-adenosylmethionine decarboxylase (SAMDC) activity. Eflornithine is safe in children (Phase 2 preventative trials in maintenance of high-risk pediatric neuroblastoma; ODC1/Bachmann-Bupp trials) and is in clinical trials and chemopreventive indications for colorectal cancer (CRC) predisposition syndrome (familial adenomatous polyposis (FAP)), certain gliomas, and prostate cancer at high-risk for invasion. Further, DFMO is FDA-approved for African trypanosomiasis and hirsutism.

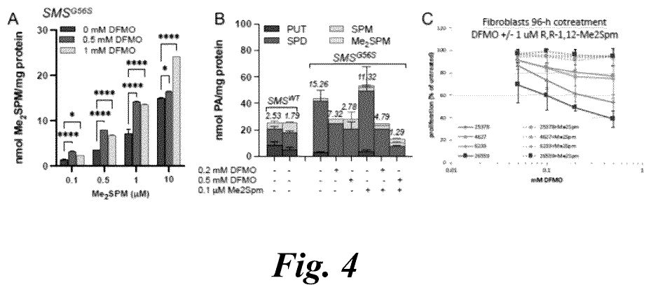

Referring now to FIG. 4A-FIG. 4C, DFMO and Me₂SPM cooperatively reduce intracellular spermidine, improve SPD/SPM ratio, and support proliferation in cells from SRS patients. As shown in FIG. 4A, DFMO increases uptake of Me₂Spm. FIG. 4B demonstrates that DFMO cooperates with Me₂Spm in improving SPD/SPM ratio, indicated by numbers above columns. As shown in FIG. 4C, Me₂Spm rescues DFMO-mediated growth inhibition.

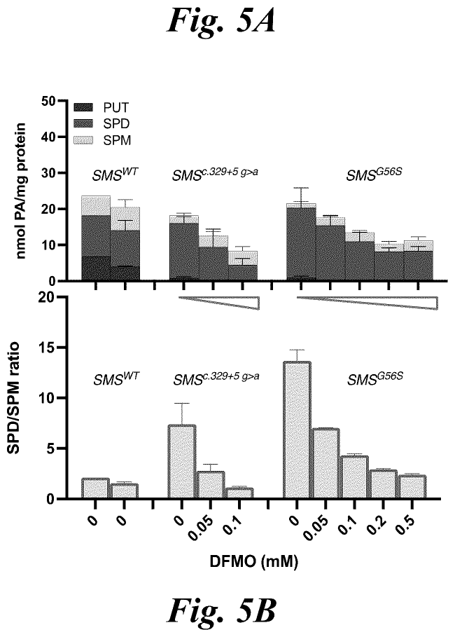

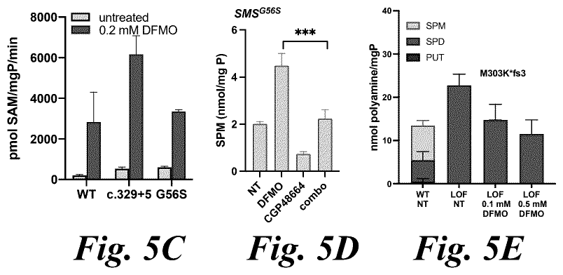

FIG. 5A-FIG. 5E demonstrate that DFMO stimulates spermine biosynthesis in hypomorphic SRS cells and show the sensitivity of DFMO stimulation and its mechanism of action. These data demonstrate that DFMO enhances conversion of SPD to SPM (FIG. 5A) and correction of the SPD/SPM ratio (FIG. 5B) in SRS patient-derived cell lines. Further, DFMO induces SAMDC activity (FIG. 5C) and cotreatment with a SAMDC inhibitor (CGP48664) prevents the conversion to SPM (FIG. 5D). These results suggest an increased availability of the aminopropyl donor dcSAM to the hypomorphic SMS enzyme as a mechanism of action. SRS cells with a complete loss-of-function mutation in SMS do not convert SPD to SPM (FIG. 5E), indicating stimulation of hypomorphic SMS enzyme activity in the response to DFMO.

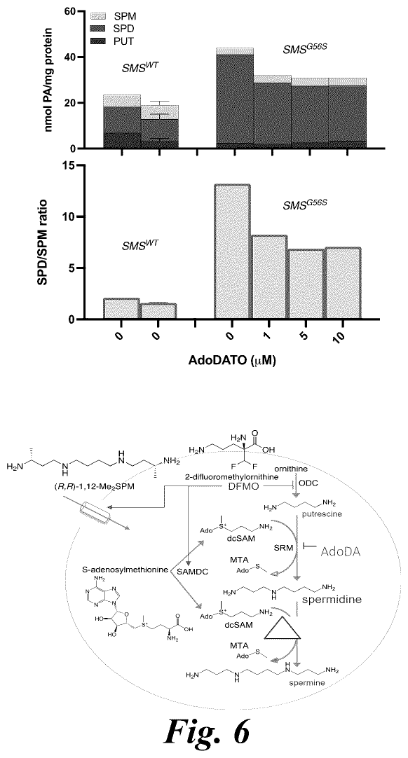

FIG. 6 demonstrates that inhibition of spermidine synthase (SRM) improves the SPD/SPM ratio while preserving putrescine pool and illustrates SRM as a second therapeutic target. AdoDATO inhibits spermidine synthase (SRM), thereby preventing SPD biosynthesis and preserving dcSAM pool for conversion of SPD to SPM by hypomorphic SMS. Treatment of SRS cells with AdoDATO improves the SPD/SPM ratio while maintaining levels of putrescine, which DFMO depletes. Maintenance of putrescine may be important in certain cell types.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Murray-Stewart, Tracy, Matthew Dunworth, Jackson R. Foley, Charles E. Schwartz, and Robert A. Casero Jr., “Polyamine Homeostasis in Snyder-Robinson Syndrome,” Medical Sciences 6, no. 4:112 (2018).
Murry Stewart, Tracy, Maxim Khomutov, Jackson R. Foley, Xin Guo, Cassandra E. Holbert, Tiffany T. Dunston, Charles E. Schwartz, Kathleen Gabrielson, Alexey Khomutov, Robert A. Casero, “(R,R)-1,12-Dimethylspermine can mitigate abnormal spermidine accumulation in Snyder-Robinson syndrome,” Journal of Biological Chemistry, 295, Issue 10:3247-3256 (2020).
U.S. Patent Application Publication No. 20210000769 for Spermine Pro-drugs, to Phanstiel et al., published Jan. 7, 2021.
RU2558953C2 for (R,R)- and (S,S)-diastereomers of 2,11-dimethylspermine and 3,10-dimethylspermine, to Khomutov et al., published Aug. 10, 2015.
Casero, Robert A Jr, and Patrick M Woster. “Recent advances in the development of polyamine analogues as antitumor agents.” Journal of medicinal chemistry vol. 52,15, 4551-73 (2009).
Bey, Philippe, F. Gerhart, V. Van Dorsselaer, and C. Danzin, α-(Fluoromethyl)dehydroornithine and α-(fluoromethyl)dehydroputrescine analogs as irreversible inhibitors of ornithine decarboxylase, Journal of Medicinal Chemistry, 26 (11), 1551-1556 (1983).
Medina-Enríquez, Miriam Marlene, Verónica Alcántara-Farfán, Leopoldo Aguilar-Faisal, José Guadalupe Trujillo-Ferrara, Lorena Rodríguez-Páez & Alba Laura Vargas-Ramírez, N-ω-chloroacetyl-1-ornithine, a new competitive inhibitor of ornithine decarboxylase, induces selective growth inhibition and cytotoxicity on human cancer cells versus normal cells, Journal of Enzyme Inhibition and Medicinal Chemistry, 30:3, 345-353 (2015).
Danzin, Charles and Patrick Casara, α-Allenyl putrescine, an enzyme-activated irreversible inhibitor of bacterial and mammalian ornithine decarboxylases, Federation of European Biochemical Societies, 1758, Volume 174, number 2, 274-278 (1984).
J. Sečkutė, McCloskey D E, Thomas H J, Secrist J A 3rd, Pegg A E, Ealick S E. Binding and inhibition of human spermidine synthase by decarboxylated S-adenosylhomocysteine. Protein Sci., 20(11):1836-44 (2011).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating a disease, condition, or disorder associated with a decrease in biosynthesis of spermine and/or an elevated spermidine/spermine level, the method comprising administering to a subject in need of treatment thereof a therapeutically effective amount of a polyamine analogue or an inhibitor of polyamine biosynthesis, such as a an ornithine decarboxylase (ODC) inhibitor or a spermidine synthase (SRM) inhibitor, or a combination thereof, to treat disease, condition, or disorder.

2. The method of claim 1, wherein the disease, condition, or disorder associated with a decrease in biosynthesis of spermine and/or an elevated spermidine/spermine level comprises Snyder-Robinson Syndrome or related disorders.

3. The method of claim 1, wherein the polyamine analogue comprises a spermine mimetic.

4. The method of claim 3, wherein the spermine mimetic is selected from (R,R)-1,12-dimethylspermine, (S,S)-1,12-dimethylspermine, (R,S)-1,12-dimethylspermine, 3,10-dimethylspermine and 2,11-dimethylspermine. See RU2558953C2 for (R,R)- and (S,S)-diastereomers of 2,11-dimethylspermine and 3,10-dimethylspermine.

5. The method of claim 4, wherein the spermine mimetic comprises 1,12-dimethylspermine.

6. The method of claim 5, wherein the 1,12-dimethylspermine comprises (R,R)-1,12-dimethylspermine.

7. The method of claim 1, wherein the inhibitor of polyamine biosynthesis comprises an ornithine decarboxylase (ODC) inhibitor selected from difluoromethylornithine (DFMO, Eflornithine), alpha-methylornithine, alpha(fluoromethyl)dehydroornithine, alpha-(fluoromethyl)dehydroputrescine, N-ω-chloroacetyl-L-ornithine, and alpha-allenyl putrescine.

8. The method of claim 1, wherein the spermidine synthase (SRM) inhibitor is selected from decarboxylated S-adenosylhomocysteine (dcSAH), bis-cyclohexylammonium sulfate (BCHS), Juglorin, adenosyl spermidine, S-adenosyl-1,8-diamino-3-thio-octane. (AdoDATO), trans-4-methylcyclohexylamine (4MCHA), cyclohexylamine (CHA), N-(3-Aminopropyl)cyclohexylamine, dicyclohexylamine (DCHA) sulfate, methylglyoxal bis-(cyclopentylamidinohydrazone) (MGBP), 2-mercaptoethylamine, 2-mercaptopropylamine, N-chlorosulfonyldicyclohexylamine, 5′-((3-aminopropyl)ammo)-5′-deoxyadenosine, 1-aminooxy-3-aminopropane, 5′-(isobutylthio)adenosine, and 5′-(methylthio)adenosine.

9. The method of claim 1, wherein administration of the polyamine analogue in combination with an inhibitor of polyamine biosynthesis or a spermidine synthase (SRM) inhibitor restores a balance of polyamine levels in the subject relative to a balance of polyamine levels in a subject not afflicted with Snyder-Robinson Syndrome or related disorders.

10. The method of claim 1, wherein administration of the poly amine analogue in combination with an inhibitor of poly amine biosynthesis results in one or more of an increase in spermine synthase (SMS) activity, a decrease in intracellular spermidine (SPD), an increase in intracellular spermine (SPM), and a decrease in SPD/SPM ratio.

11. A composition comprising a polyamine analogue and an inhibitor of polyamine biosynthesis and a pharmaceutically acceptable excipient.
